(19) United States
(12) Patent Application Publication
(10) Pub. No.: US 2013/0310530 A1
(43) Pub. Date: Nov. 21, 2013

(54) USE OF SILICONE METHACRYLATE PARTICLES IN COSMETIC FORMULATIONS

(71) Applicants: Brajesh Kumar Jha, Midlothian, VA (US); Jürgen Meyer, Essen (DE); Matthias Naumann, Hamburg (DE); Joachim Venzmer, Essen (DE); Susann Wiechers, Essen (DE)

(72) Inventors: Brajesh Kumar Jha, Midlothian, VA (US); Jürgen Meyer, Essen (DE); Matthias Naumann, Hamburg (DE); Joachim Venzmer, Essen (DE); Susann Wiechers, Essen (DE)

(73) Assignee: EVONIK GOLDSCHMIDT GMBH, Essen (DE)

(21) Appl. No.: 13/981,366

(22) PCT Filed: Dec. 21, 2011
(86) PCT No.: PCT/EP2011/073544

§ 371 Date: Jul. 24, 2013

(30) Foreign Application Priority Data
Jan. 25, 2011 (DE) 102011003090.5

(51) Int. Cl. A61K 8/81

(52) U.S. Cl. CPC A61K 8/8152
USPC 526/279

(57) ABSTRACT

The present invention relates to cosmetic compositions comprising, as solid particles, silicone methacrylate particles which are obtainable by the steps a) producing an emulsion of water and an organic phase, where the organic phase comprises specifically organopolysiloxanes modified in the terminal and/or lateral position with methacrylate groups, or mixtures thereof, with the addition of at least one emulsifier and optionally one or more coemulsifiers, where the organic phase forms the internal phase of the emulsion, and fully polymerizing the internal phase in the presence of a radical initiator, which is added to the external phase (aqueous phase) in a concentration of from 0.1 to 40% by weight based on the internal phase, and also to corresponding cosmetic compositions.

The present invention relates to cosmetic compositions comprising solid particles characterized in that the solid particles used are silicone methacrylate particles which are obtainable by the steps a) producing an emulsion of water and an organic phase, where the organic phase comprises specifically organopolysiloxanes modified in the terminal and/or lateral position with methacrylate groups, or mixtures thereof, with the addition of at least one emulsifier and optionally one or more coemulsifiers, where the organic phase forms the internal phase of the emulsion, and fully polymerizing the internal phase in the presence of a radical initiator, which is added to the external phase (aqueous phase) in a concentration of from 0.1 to 40% by weight based on the internal phase.

It is known to use hydrophobic or hydrophobicized particles or silicone resin particles in cosmetic, in particular make-up, formulations, in order to improve the durability of the make-up on the skin. For this, silicone-gum powders or powders of silicone elastomers are often used.

EP 0 834 305 describes gel-like cosmetic skin-treatment compositions which comprise spherical powders of organopolysiloxane elastomers having an average particle size of from 1 to 15 μm. As further particles, in particular hydrophobicized or silicone-treated inorganic powders are added.

EP 0 765 656 describes cosmetic, water-in-oil emulsions as cosmetic compositions which comprise powders of spherical, elastomeric or organopolysiloxane particles. Besides the elastomeric (deformable) particles, these compositions have hydrophobicized silica particles.

FR 2682534 describes skin cosmetics which have two different particle fractions, where one fraction consists of non-deformable particles, preferably glass beads and the other consists of deformable, i.e. elastic. particles.

It is a common feature of the cited documents that, by using the elastomer particles, a pleasant (soft) powdery skin feel should be achieved.

Disadvantages of using elastomeric particles are often the content of silicone oil present in the silicone elastomer particles, which is undesired in certain cosmetic formulations and, in the event of escape, can adversely affect the composition and stability of the formulation, and also the high costs associated with production and the limited formulation scope.

The object of the present invention was to provide cosmetic compositions which comprise particles which do no have one or more of the specified disadvantages of the elastomeric particles.

Surprisingly, it has been found that the use of silicone methacrylate particles, described below, in cosmetic compositions makes accessible skin cosmetics which impart a pleasant, velvety, powdery skin feel, which corresponds to the skin feel of typical silicone elastomer-containing compounds, without the particles used being silicone elastomer particles and/or having their disadvantages.

The invention therefore provides a composition as defined in Claim 1, comprising solid particles, which is characterized in that, as solid particles, silicone methacrylate particles are present which are obtainable by the steps: a) producing an emulsion of water and an organic phase, where the organic phase comprises organopolysiloxanes modified in the terminal and/or lateral position with methacrylate groups according to the general formula (I) or mixtures thereof, with the proviso that if b and c=0, R³ must not be selected from the same group as R¹, with the addition of at least one emulsifier and optionally one or more coemulsifiers, where the organic phase forms the internal phase of the emulsion, and b) fully polymerizing the internal phase in the presence of a radical initiator, which is added to the external phase (aqueous phase) in a concentration of from 0.1 to 40% by weight based on the internal phase.

Compared to the silicone acrylate particles described in DE 10 2007 058 713, the silicone methacrylate particles used according to the invention have the advantage that cosmetics produced with them have a considerably higher storage stability. Even in the unprocessed state, after storage for several weeks at room temperature, the silicone acrylate particles have a clearly perceptible odour of acrylic acid, which is formed as a result of hydrolysis with the atmospheric moisture. This effect likewise arises in a formulation containing water, which is described in the examples.

Moreover, the use according to the invention of the silicone methacrylate particles for producing cosmetic compositions (emulsions) has the advantage that these have a stabilizing effect. Thus, compositions according to the invention which comprise the silicone methacrylate particles can have a better storage stability, but also have a higher stability upon passing through freeze-thaw cycles.

On account of the stabilizing effect of the silicone methacrylate particles used, they can be used to produce stable cosmetic compositions (emulsions) without having to use the otherwise required amount of emulsifier. Within the context of the present invention, stable emulsions are understood as meaning those emulsions of water and oil which, even after storage for 3 months at 25° C., exhibit no separation into oil phase and water phase that is visible to the naked eye.

The compositions according to the invention and the use according to the invention of silicone methacrylate particles are described below by way of example without any intention of limiting the invention to these exemplary embodiments.

Where ranges, general formulae or compound classes are given below, then these are intended to encompass not only the corresponding ranges or groups of compounds that are explicitly mentioned, but also all part ranges and part groups of compounds which can be obtained by removing individual values (ranges) or compounds. Where documents are cited in the course of the present description, then their content, in its entirety, should be deemed as forming part of the disclosure of the present invention. Where, within the context of the present invention, compounds such as e.g. organomodified polysiloxanes are described which can have a plurality of different units, then these may occur in random distribution (random oligomer or polymer) or in an arranged manner (block oligomer or block polymer) in these compounds. Unless stated otherwise, average data are number averages, and percentages are in mass %.

Cosmetic compositions according to the invention comprising solid particles are characterized in that the composition has, as solid particles, at least silicone methacrylate particles which are obtainable by the steps

a) producing an emulsion of water and an organic phase, where the organic phase comprises organopolysiloxanes modified in the terminal and/or lateral position with methacrylate groups according to the general formula (I) or mixtures thereof

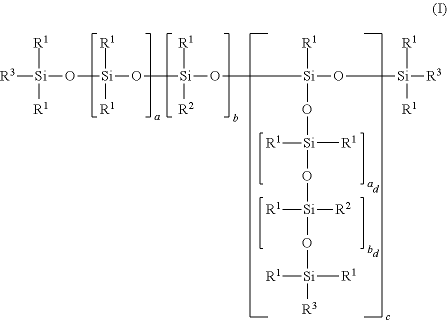

Where

R¹=identical or different radicals, selected from linear or branched, saturated, mono- or polyunsaturated, linear, cyclic or branched alkyl, alkoxy, polyalkoxy, hydroxyalkyl, hydroxyalkoxy, alkenyl, aryl, aryloxy, hydroxyaryl, hydroxyaryloxy, alkaryl, alkaryloxy, hydroxyalkaryl, hydroxyalkaryloxy, aralkyl, aralkoxy, hydroxyaralkyl or hydroxyaralkoxy radicals having 1 to 20 carbon atoms and optionally containing one or more ether or ester bridges, preferably identical or different radicals, selected from linear or branched, saturated, mono- or polyunsaturated linear, cyclic or branched alkyl, aryl, alkaryl or aralkyl radicals having 1 to 20 carbon atoms and optionally containing one or more ether or ester bridges, preferably methyl radical,

R²=identical or different divalent hydrocarbon radicals having 1 to 20 carbon atoms bonded to the Si atom via an Si—C linkage and to which at least one methacrylic acid unit is attached via an ester bond,

R³=identical or different radicals R¹ or R², preferably R²,

a=50 to 1000, preferably 100 to 210, particularly preferably 140 to 190,

b=0 to 15, preferably 5 to 10,

c=0 to 5, preferably 0,

a_{d}=0 to 1000,

b_{d}=0 to 15,

where

the index d when c>0 is an integer>0, with the proviso that when b and c=0, R³ must not be selected from the same group as R¹, with the addition of at least one emulsifier, preferably a solid emulsifier (particulate emulsifier), and optionally one or more coemulsifiers, where the organic phase forms the internal phase of the emulsion, and b) fully polymerizing the internal phase in the presence of a radical initiator, which is added to the external phase (aqueous phase) in a concentration of preferably from 0.1 to 40% by weight, preferably 0.5 to 25% by weight and particularly preferably 10 to 15% by weight, based on the internal phase. The full polymerization thus preferably takes place in the form of a suspension polymerization.

The numerical values for a, b and c are preferably statistical (numerical) average values. The index d is a whole-numbered index term (run variable).

The silicone methacrylate particles used according to the invention are preferably not elastic or are composed essentially (more than 90 mass %) of non-elastic material. Within the context of the present invention, non-elastic material is understood as meaning a material which, polymerized in bulk, upon testing based on DIN 53 504, cannot be extended to 100% without tearing, preferably not to 50% and particularly preferably not to 25%.

In the cosmetic composition according to the invention, preference is given to using silicone methacrylate particles which have a maximum of the particle size distribution in the range from 1 to 50 μm, preferably from 5 to 20 μm. By using the silicone methacrylate particles in the specified particle size range, a pleasant, velvet-silky, powdery skin feel is attained. If the maximum of the particle size distribution is at larger particle sizes, a more abrasive skin feel is obtained, and smaller particle sizes either have no sensory effect or produce a rather rough skin feel.

The particle size distribution of the dry silicone methacrylate particles can be determined using a measuring system such as, for example, from Sympatec, consisting of the modules VIBRI, RODOS and HELOS. The particles are predispersed by vibration and dispersed by means of compressed air, preferably using an overpressure of from 2 to 3 bar, and blown through a measuring chamber. Therein, the particle size distribution is determined by means of laser diffraction. The evaluation takes place with the associated WINDOX software.

Preferred cosmetic compositions according to the invention are those which have a fraction of silicone methacrylate particles in the overall composition of from 0.01 to 80% by weight, preferably from 0.1 to 10% by weight.

As further components, the compositions according to the invention can have those as are customary in cosmetic compositions. Thus, for example, the compositions according to the invention can comprise one or more additional components selected from the group of

emollients,

emulsifiers,

thickeners/viscosity regulators/stabilizers

antioxidants,

hydrotropes (or polyols),

solids and fillers,

pearlescent additives,

deodorant and antiperspirant active ingredients,

insect repellents,

self-tanning agents,

preservatives,

conditioners,

perfumes,

dyes,

cosmetic active ingredients,

care additives,

superfatting agents,

solvents.

Substances which may be present as exemplary representatives of the individual groups are known to the person skilled in the art and can be found, for example in the German application DE 102008001788.4. This patent application is hereby incorporated by reference and thus forms part of the disclosure.

As regards further optional components, and also the amounts used of these components, reference is made expressly to the relevant handbooks known to the person skilled in the art, for example K. Schrader, “Grundlagen and Rezepturen der Kosmetika”, [Fundamentals and Formulations of Cosmetics”], 2nd edition, pages 329 to 341, Hüthig Buch Verlag Heidelberg.

The amounts of the particular additives are governed by the intended use.

Typical guide formulations for the respective applications are known prior art and are contained, for example, in the brochures of the manufacturers of the particular basic materials and active ingredients. These existing formulations can usually be adopted unchanged. If necessary, however, for the purposes of adaptation and optimization, the desired modifications, can be undertaken by simple experiments without complications.

The compositions according to the invention particularly preferably have one or more of the components listed below:

antimicrobial substances and/or preservatives, such as triclosan and triclocarban and hexachlorophene, complexing agents such as, for example EDTA (acid and salts), citric acids and etidronic acid and salts thereof, UV absorbers, such as, for example, derivatives of benzophenone, of benzotriazole, cinnamic acid esters or particulate UV absorbers such as for example, ZnO or TiO₂, dyes and colorants, pigments, spraying auxiliaries, wetting agents, vitamins, growth promoters, hormones and also fragrances.

It may be advantageous if the composition according to the invention has from 0.1 to 20% by weight based on the total composition, of components which absorb or filter out UV radiation. Such substances can be e.g. the UV absorbers specified above.

The cosmetic compositions (emulsions) according to the invention can comprise further solids (cosmetic particles) which are different from the specified silicone methacrylate particles, in particular pigments such as titanium dioxide, zinc oxide or iron oxide, viscosity regulators such as pyrogenic or precipitated silica or other particles. Preferred compositions according to the invention have from 0.1 to 15% by weight of particles which are different from the stated silicone methacrylate particles. The silicone methacrylate particles used according to the invention make it possible here to conceal the rough skin feel of these other pigments (cosmetic particles) in the compositions by means of a powdery velvety skin feel. Particular preference is given therefore to the use of the silicone methacrylate particles in sunscreen products, make-up products, lipsticks and other cosmetic products for decorative applications.

The further solids (particles) used which are different from the specified silicone methacrylate particles preferably have a particle size of from 5 to 50 μm and can be used e.g. for achieving an improved skin feel, a matting or for visually reducing wrinkles (“soft focus effect”). Typical particle materials are, e.g. PMMA, PS, PE, PP, nylon, in particular nylon-12 and nylon-6, silicone particles and silicone elastomers, starch, talc, mica and boron nitride. Depending on the profile of properties, the particles used here can either have a compact structure or a porous structure. Within the context of the invention, further solids (cosmetic particles) which may be present in the compositions according to the invention are also silicone elastomer gels. The silicone elastomer gels are preferably silicone elastomers which have been produced directly in a carrier oil. The carrier oils are preferably typical cosmetic oils, such as, for example, cyclic or linear silicone oils, mineral oils, ester oils, ether oils or triglycerides. Typical silicone elastomer gels are, for example, Dow Corning 9040 Silicone Elastomer Blend and Dow Corning 9041 Silicone Elastomer Blend (Dow Corning) or the products KSG-15 or KSG-18 (Shin Etsu).

It may be advantageous if the compositions according to the invention have from 0.1 to 20% by weight of organosilicon compounds. The organosilicon compounds here may be present e.g. as emulsifiers or as emollient in the composition according to the invention. Suitable organosilicon compounds are, for example cyclic or linear alkylsiloxanes, in particular methylsiloxanes, such as, e.g. dimethicone, D4- or D5-cycles, which are preferably present as emollient in the composition according to the invention, or organomodified siloxanes, in particular polyether-modified polysiloxanes. Such organomodified siloxanes are preferably present as emulsifiers in the composition according to the invention.

Further specific organosilicon compounds are in particular cosmetic emulsifiers for O/W and W/O emulsions, these preferably being: polysiloxane-polyether copolymers (dimethicone copolyols), such as e.g. PEG/PPG-20/6 dimethicone, PEG/PPG-20/20 dimethicone, bis-PEG/PPG-20/20 dimethicone, PEG-12 or PEG-14 dimethicone, PEG/PPG-14/4 or 4/12 or 20/20 or 18/18 or 17/18 or 15/15, also alkoxylated polysiloxane-polyether copolymers such as, for example, methoxy PEG/PPG-25/4 dimethicone. Furthermore, also terminally modified silicone polyethers such as, for example, bis-PEG/PPG-14/14-dimethicone; polysiloxane-polyalkyl-polyether copolymers and corresponding derivatives, such as, e.g. lauryl or cetyl dimethicone copolyols, in particular cetyl PEG/PPG-10/1 dimethicone (ABIL® EM 90 (Evonik Goldschmidt)); polyether siloxanes, which contain both comb-position and terminal-position modification, such as e.g. bis-PEG/PPG-20/5 PEG/PPG-20/5 dimethicone or bis-PEG/PPG-16/16 PEG/PPG-16/16 dimethicone; PEG-free silicone emulsifiers, such as e.g. lauryl glyceryl dimethicone, lauryl polyglyceryl-3 polydimethylsiloxyethyl dimethicone, polyglyceryl-3 disiloxane dimethicone or amodimethicone glycerocarbamate.

The cosmetic compositions according to the invention comprising silicone methacrylate particles are preferably cosmetic emulsions. These may either be of the water-in-oil (W/O) type or of the oil-in-water (O/W) type. Preferably, the oil phase of these emulsions can comprise one or more silicone oils, and in some cases, these emulsions can comprise exclusively silicone oils as oils. In this case, these emulsions according to the invention can also be referred to as W/Si or Si/W emulsions. Particularly preferred silicone oils here are liquid cyclomethicones or dimethicones with a viscosity of from 1 to 200 mPas.

Particularly preferred compositions according to the invention have, in each case based on the total composition, from 0.1 to 20% by weight of components, which absorb or filter out UV radiation, from 0.1 to 15% by weight of particles which are different from the specified silicone methacrylate particles, and from 0.1 to 20% by weight of organosilicon compounds.

The process for the preparation of the silicone methacrylate particles can be carried out in principle as described in DE 10 2007 058 713 in general terms for silicone (meth)acrylate particles, to which reference is made in its entirety.

Emulsifiers which can be used for producing the silicone methacrylate particles are all customary emulsifiers. These may be anionic, cationic or non-ionic surface-active substances.

Typical emulsifiers are, e.g. alkyl sulphates, preferably having a chain length of 10 to 18 carbon atoms, alkyl and aryl ether sulphates, preferably having 10 to 24 carbon atoms in the hydrophobic radical and having preferably up to 40 ethylene oxide or propylene oxide units, alkyl and alkylarylsulphonates having preferably 10 to 24 carbon atoms, alkyldiphenyl oxide disulphonates, oleic acid sulphonates, esters and half-esters of sulphosuccinic acid with monohydric alcohols or alkylphenols, alkyl and alkenyl carboxylates having preferably a chain length of 10 to 18 carbon atoms, alkyl polyglycol ethers and alkylaryl polyglycol ethers having preferably in each case 4 to 40 ethylene oxide units, alkyl and alkenyl alcohols having preferably 12 to 20 carbon atoms, ethoxylated alkyl and alkenyl alcohols having preferably 12 to 20 carbon atoms and ethoxylated alkylphenols. Of suitability for cosmetic applications are in particular emulsifier systems which usually serve for the emulsification of silicone oils, as supplied, for example, by Evonik Goldschmidt GmbH under the names ABIL® EM 90, ABIL® EM 97 or ABIL® Care XL 80. In particular, emulsifiers and surfactants known from cosmetic applications can be used, as are listed, for example, in DE 10 2005 011785 A1.

It may be advantageous if, in step a) a solids-stabilized emulsion is produced. For this, the particulate emulsifiers which can be used are particles which are particularly preferably selected from the group of metal oxides, mixed oxides, nitrides, hydroxides, carbonates, silicates, silicone resins, silicones and/or organic polymers, which are preferably at least (partially) hydrophobicized, e.g. with at least one compound from the group of silanes, siloxanes, quaternary ammonium compounds, cationic polymers and fatty acids or anions thereof. Particularly preferably used particulate emulsifiers are, for example, colloidal silica particles, which are available under the trade name LUDOX® from Grace Davidson.

The particulate emulsifiers can be used in the process according to the invention as they are or in the form of dispersions or sols, in particular aqueous dispersions or sols. It may be advantageous if particulate emulsifiers are used which preferably have an average primary particle size in all dimensions of greater than 0.1 to 1 μm.

The determination of the primary particle size can be determined e.g. by visual evaluation of an image generated by transmission electron microscopy.

Particularly when using particulate emulsifiers, it may be advantageous if, in step a) of the process according to the invention, the preparation of the emulsion is carried out with the addition of one or more coemulsifiers. Coemulsifiers which can be used in the process according to the invention are, in particular those compounds which interact with the solid-state emulsifier particles, preferably those which attach to hydrophobicizing solid-state emulsifier particles. In the process according to the invention, coemulsifiers which can be used are in particular compounds selected from the group of cationic surfactants. Cationic coemulsifiers which can be used are in particular cationic ammonium compounds. Such compounds are available, e.g. under the trade names VARISOFT® 470 P, VARISOFT® TC-90, VARISOFT® 110, VARISOFT® TA-100, ADOGEN® 442-100 P, ADOGEN® 432, ADOGEN® 470, ADOGEN® 471, ADOGEN® 464, VARIQUAT® K 300, VARIQUAT® B 343, VARIQUAT® 80 ME, REWOQUAT® 3690, REWOQUAT® WE 15, REWOQUAT® WE18, REWOQUAT® WE 28 or REWOQUAT® CR 3099 from Evonik Goldschmidt GmbH. In the process according to the invention, preference is given to using VARISOFT® TA-100 or VARISOFT® PATC (both available from Evonik Goldschmidt GmbH), and particular preference is given to using VARISOFT® PATC as cationic coemulsifier. In the process according to the invention, very particular preference is given to using those silicone methacrylate particles for whose preparation no cetyltrimethylammonium bromide is/has been used.

In the process, the silicone methacrylates of the formula (I) used are preferably those in which more than 70 mol %, particularly preferably more than 95%, very particularly preferably all of the radicals R¹ in formula (I) are methyl groups.

The radicals R² in the general formula (I) are preferably selected from the group of the radicals

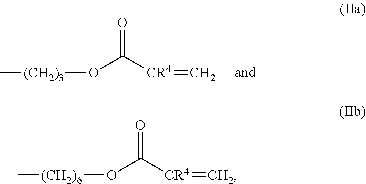

where R⁴ is a methyl group.

It may be particularly advantageous to use those silicone methacrylate particles for whose preparation silicone methacrylates of the formula (I) are used in which a assumes a value from 100 to 210, preferably from 140 to 190 and b assumes a value from 3 to 9 and c=0, as described in the patent specification DE 3810140.

In the process for the preparation of the silicone methacrylate particles, the silicone methacrylates of the formula (I) can be used individually or as mixtures, in particular as statistical mixtures. In the process according to the invention, preference is given to using mixtures of silicone methacrylates of the formula (I), in which the silicone methacrylates differ with regard to their structure and/or their molecular weight.

For further details relating to the preparation process of the silicone methacrylate particles, and also for preferred embodiments of the process for the preparation of silicone methacrylate particles, reference is made explicitly to the description in DE 10 2007 058 713 A1.

The preparation of the compositions/dispersions using the silicone methacrylate particles can take place by means of customary methods corresponding to the prior art, although the particles which are formed after the polymerization according to step b) of the preparation process of the particles and washing with alcohols and/or water can also be further processed without prior drying to give, for example aqueous dispersions. This is also possible if, for example, a desired surface modification can take place directly from aqueous or alcoholic phase, which has a favourable effect on the process economics.

The silicone methacrylate particles according to the invention can be incorporated during the preparation of cosmetic emulsions either directly into the hot or cold oil phase, or they can, following combination of water phase and oil phase to give a W/O or O/W emulsion, preferably at temperatures of less than 50° C., be incorporated into the already formed emulsion.

The cosmetic compositions according to the invention are preferably a skincare composition, a make-up or a sunscreen composition.

Moreover, the present invention provides the use of silicone methacrylate particles which are obtainable by the process described above, involving steps a) and b), for producing cosmetic compositions comprising solid particles. Preference is given to using the silicone methacrylate particles described above as preferred and/or those prepared by the preferred process. In particular, those silicone methacrylate particles are used which have a maximum of the particle size distribution in the range from 1 to 20 μm, and/or have been obtained by producing a solids-stabilized emulsion in step a), where the emulsifiers used are particulate emulsifiers which are selected from the group of metal oxides, mixed oxides, nitrides, hydroxides, carbonates, silicates, silicone resins, silicones and/or organic polymers which are at least (partially) hydrophobicized and with at least one compound from the group of silanes, siloxanes, quaternary ammonium compounds, cationic polymers and fatty acids or anions thereof, and/or in which more than 95 mol % of the radicals R¹ in formula (I) are methyl groups, and/or in which R² in the general formula (I) is selected from the group of the radicals

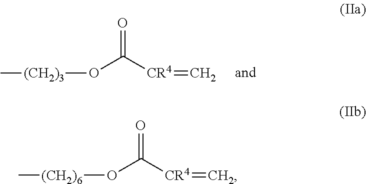

where R⁴ is a methyl group and/or during whose preparation silicone methacrylates of the formula (I) are used, in which a assumes a value from 150 to 210 and b assumes a value from 3 to 9 and c=0.

In the examples listed below, the present invention is described by way of example without any intention of limiting the invention, the scope of application of which arises from the entire description and the claims, to the embodiments specified in the examples. Where percentages are specified in the following examples, unless stated otherwise, these are percentages by mass.

The formulation examples listed below are for the most part emulsions of the oil-in-water (O/W) type or water-in-oil (W/O) type. These can be prepared by customary methods known to the person skilled in the art using typical stirring units. Preferably, W/O emulsions are prepared by slowly stirring the water phase (B) into the oil phase (A) with subsequent homogenization. For the described O/W emulsions, preferably oil phase and water phase are brought together without stirring and then homogenized. This can take place in a cold/cold process (Example 3) or in a hot/hot process, in which the homogenization takes place at ca. 70° C. (Example 2). The silicone methacrylate particles according to the invention can here in principle be incorporated in any stage of the process. In most example formulations, either the incorporation took place into the finished emulsion at temperatures of <40° C. or the silicone methacrylate particles were initially introduced directly at the start of the emulsion preparation together with the oil phase.

The nomenclature relating to the topic “stability” used for evaluating the emulsion comparative examples is based on the following requirements. If the stability is assessed as “good”, then this means that such an emulsion is stable for at least one month at room temperature, −5° C. and 40° C. “Stable” here means that no kind of oil or water separation occurs, that the appearance of the emulsion remains homogeneous and that no noteworthy changes in viscosity, colour or odour arise in the emulsion.

The skin feel of the cosmetic formulations described in the examples below was determined by a so-called panel. At least five people compared the sensory properties of the cosmetic formulations and the respective comparison formulation without knowing the composition. The properties listed are those which the majority of the people described with preference.

### EXAMPLE 1

### Particles of Silicone Methacrylate

588.5 g of dipotassium hydrogenphosphate were dissolved in 550 g of demineralized water. 2600 g of demineralized water and 115 g of Ludox® SM were mixed and adjusted to pH=7. With stirring, 1100 g of silicone methacrylate (described in Patent Specification DE 3810140) were added and the mixture was pre-emulsified for several minutes. Then, with stirring, 27.5 g of a 5% strength aqueous solution of VARISOFT® PATC were added and the mixture was pre-emulsified again for a few minutes. The mixture produced in this way was then homogenized using a homogenizer of the Microfluidizer type from Microfludics with an interaction chamber of diameter 200 μm at 300 bar.

For the polymerization, the emulsion was placed in a round flask and flushed with nitrogen with stirring and heated to 80° C. Firstly, 153 g of ammonium peroxodisulphate dissolved in 500 g of demineralized water and then 93.87 g of 38% strength sodium hydrogen sulphite solution were added. The reaction mixture was stirred for three hours at 80° C. The finished dispersion was then admixed with 3% of 30% strength aqueous hydrogen peroxide solution in terms of the entire amount of the mixture and left to stand for 2-3 hours and cooled to room temperature. The particles were purified by means of repeated (at least three times) washing with demineralized water. The reaction mixture was filtered off with suction over a suction filter and slurried with water, and the water was again filtered off with suction. Drying was carried out in a drying cabinet at 50-65° C. at atmospheric pressure—if necessary additionally in vacuo at 40° C., to constant weight.

### EXAMPLE 2 AND COMPARATIVE EXAMPLE C2

The formulations given in Table 1 were prepared, and their stability, appearance and skin feel were assessed.

TABLE 1
Formulations and Results of Example 2 and Comparative Example C2, oil-
in-water care cream

| | | Example | |
|---|---|---|---|
| | | 2 | C2 |
| A | TEGO ® Care 165 | 6.0% | 6.0% |
| | (Evonik Goldschmidt GmbH) | | |
| | (glyceryl stearate; | | |
| | PEG-100 stearate) | | |
| | Stearyl alcohol | 3.0% | 3.0% |
| | Mineral oil | 4.0% | 4.0% |
| | Ethylhexyl palmitate | 4.0% | 4.0% |
| B | Glycerol | 3.0% | 3.0% |
| | Water | 75.0% | 80.0% |
| C | Silicone methacrylate particles | 5.0% | |
| | from Ex. 1 | | |
| Z | Methylparaben, ethylparaben, | q.a. | q.a. |
| | methylisothiazolinone, perfume | | |
| | Stability | good | good |
| | Appearance | white, | white, |
| | | homogeneous | homogeneous |
| | Skin feel | velvety-silky, | waxy, |
| | | smooth; | not smooth |
| | | not dull | |

### EXAMPLE 3 AND COMPARATIVE EXAMPLE C3

### Oil-in-Water Body Care Lotions Prepared Under Cold Conditions

The formulations given in Table 2 were prepared, and their stability, appearance and skin feel were assessed.

TABLE 2
Formulation and Results of the oil-in-water body care lotion
prepared under cold conditions in Example 3:

| | | Example |
|---|---|---|
| | | 3 |
| A | TEGO ® Care LTP | 1.5% |
| | (Evonik Goldschmidt GmbH) | |
| | Sorbitan laurate; | |
| | polyglyceryl-4 laurate; | |
| | dilauryl citrate) | |
| | Cyclopentasiloxane | 10.0% |
| | Isohexadecane | 3.5% |
| | Ethylhexyl palmitate | 1.1% |
| | TEGO ® Carbomer 140 | 0.15% |
| | (Evonik Degussa GmbH) | |
| | TEGO ® Carbomer 141 | 0.15% |
| | (Evonik Degussa GmbH) | |
| | Xanthan | 0.1% |
| B | Glycerol | 3.0% |
| | Water | 79.6% |
| C | NaOH (10% strength solution) | 0.90% |
| D | Silicone methacrylate particles from Ex. 1 | 5.0% |
| Z | Phenoxyethanol, benzoic acid, dehydroacetic | q.a. |
| | acid, ethylhexylglycerol, polyaminopropyl | |
| | biguanide, perfume | |
| | Stability | good |
| | Appearance | white, homogeneous |
| | Skin feel | light; velvety; smooth |

### EXAMPLE 4 AND COMPARATIVE EXAMPLE C4

### Water-in-Oil Foundation

The formulations given in Table 3 were prepared, and their stability, appearance and skin feel were assessed.

TABLE 3
Water-in-oil foundation according to
Example 4 and Comparative Example C4:

| | | Example | |
|---|---|---|---|
| | | 4 | C4 |
| A | ABIL ® EM 90 | 3.0% | 3.0% |
| | (Evonik Goldschmidt GmbH) | | |
| | (Cetyl PEG/PPG-10/1 | | |
| | dimethicone) | | |
| | Diethylhexyl carbonate | 10.0% | 10.0% |
| | Cyclopentasiloxane | 7.6% | 7.6% |
| | Ethylhexyl palmitate | 3.4% | 3.4% |
| | Iron oxide | 1.8% | 1.8% |
| | Titanium dioxide | 7.2% | 7.2% |
| | Talc | 2.0% | 2.0% |
| | Silicone methacrylate particles | 2.5% | |
| | from Ex. 1 | | |
| B | NaCl | 1.0% | 1.0% |
| | Glycerol | 2.0% | 2.0% |
| | Water | 65.5% | 68.0% |
| Z | Phenoxyethanol; | q.a. | q.a. |
| | methylparaben; ethylparaben, | | |
| | butylparaben; propylparaben, | | |
| | isobutylparaben, perfume | | |
| | Stability | good | good |
| | Appearance | homogeneous, | homogeneous, |
| | | brownish | brownish |
| | Skin feel | smooth, not dull, | somewhat dry and |
| | | velvety | not smooth |

### EXAMPLES 5 AND C5

### Example of Odour Development when Using Particles of Silicone Acrylate TEGO® RC 726

The formulations given in Table 4 were prepared, and their stability, appearance and skin feel were assessed.

TABLE 4
Oil-in-water sunscreen lotion according to Example 5 and C5.

| | | Example | |
|---|---|---|---|
| | | 5 | C5 |
| A | AXOL ® C 62 | 2.00% | 2.00% |
| | (Evonik Goldschmidt GmbH) | | |
| | (Glyceryl stearate citrate) | | |
| | Cetearyl alcohol | 1.00% | 1.00% |
| | C₁₂₋₁₅ alkyl benzoate | 8.00% | 8.00% |
| | Triisostearin | 1.00% | 1.00% |
| | Diethylhexyl carbonate | 2.75% | 2.75% |
| | Tocopheryl acetate | 0.50% | 0.50% |
| | Xanthan | 0.40% | 0.40% |
| | Ethylhexyl methoxycinnamate | 7.00% | 7.00% |
| | Butyl methoxydibenzoylmethane | 3.00% | 3.00% |
| | TEGO ® Sun T 805 | 2.25% | 2.25% |
| | (Evonik Goldschmidt GmbH) | | |
| | (Titanium dioxide; | | |
| | trimethoxycaprylylsilane) | | |
| | Silicone acrylate particles of | | 2.50% |
| | TEGO ® RC 726 | | |
| | (see DE 200710058713) | | |
| | Silicone methacrylate particles | 2.50% | |
| | from Ex. 1 | | |
| B | Glycerol | 2.00% | 2.00% |
| | Water | 67.60% | 67.60% |
| Z | Phenoxyethanol, | q.a. | q.a. |
| | ethylhexylglycerol, perfume | | |
| | Stability | good | good |
| | Appearance | white, | white, |
| | | homogeneous | homogeneous |
| | Skin feel | caring, | caring, |
| | | smooth, | smooth, |
| | | velvety | velvety |
| | Odour after storage for two | no odour | significant |
| | weeks at room temperature | | odour |
| | | | reminiscent |
| | | | of acrylic |
| | | | acid and |
| | | | acrylate |
| | | | compounds |

### COMPARATIVE EXAMPLE C6

### Dry Storage of Silicone Acrylate Particles of TEGO® RC 726

The dry particles were stored in a screw-top jar for several weeks at room temperature. After four weeks, a slight odour reminiscent of acrylic acid could be detected, which became even stronger in the following weeks.

The application examples show that the silicone methacrylate particles according to the invention can be incorporated into cosmetic formulations. Using these particles significantly improves the sensory properties of cosmetic formulations without adversely affecting stability and the appearance of the example emulsions. In particular, the incorporation of the particles leads to a more velvety, more silky, less dry and less rough skin feel.

In particular, the silicone methacrylate particles are also suitable for being used in formulations together with pigments since they considerably improve the usually somewhat rough skin feel of pigment-containing formulations.

Silicone methacrylate particles have the advantage over silicone acrylate particles of not developing an unpleasant odour reminiscent of acrylic acid or acrylate compounds, which makes the latter unsuitable particularly for use in cosmetic formulations.

### EXAMPLES 7 AND C7

### Emulsion-Stabilizing Effect of the Silicone Methacrylate Particles According to the Invention

As well as the sensorily achievable advantages already described, it was also found that the silicone methacrylate particles according to the invention can improve the stability of cosmetic formulations. In particular, this effect was evident in W/O emulsions. Comparative Example C7 shows a W/O emulsion with photoprotective filters which exhibits oil deposition at room temperature and at elevated temperature. The addition of 2% silicone methacrylate particles according to the invention leads to stable emulsions with an overall constant oil phase content.

TABLE 5
Water-in-oil emulsion with photoprotective filters according to Example 7
and Comparative Example C7:

| | | Example | |
|---|---|---|---|
| | | 7 | C7 |
| A | ABIL ® EM 90 | 1.5% | 1.5% |
| | (Evonik Goldschmidt GmbH) | | |
| | (Cetyl PEG/PPG-10/1 dimethicone) | | |
| | ABIL ® EM 97 S | 1.5% | 1.5% |
| | (Evonik Goldschmidt GmbH) | | |
| | (Bis-PEG/PPG-14/14 dimethicone; | | |
| | dimethicone) | | |
| | Diethylhexyl carbonate | 10.0% | 11.0% |
| | Cyclopentasiloxane | 10.0% | 11.0% |
| | Butyl methoxydibenzoylmethane | 1.0% | 1.0% |
| | Bis-Ethylhexyloxyphenol | 1.0% | 1.0% |
| | methoxyphenyl triazine | | |
| | Octocrylene | 4.0% | 4.0% |
| | Ethylhexyl salicylate | 4.0% | 4.0% |
| | Ethylhexyl triazone | 0.5% | 0.5% |
| | Silicone methacrylate particles from | 2.0% | |
| | Ex. 1 | | |
| B | NaCl | 1.0% | 1.0% |
| | Glycerol | 2.0% | 2.0% |
| | Water | 60.8% | 60.8% |
| Z | Euxyl PE 9010 (Schülke) | 0.7% | 0.7% |
| | (phenoxyethanol; ethylhexylglycerol | | |
| | Stability | good | oil separation |
| | | | at RT and 40° C. |

### Further Formulation Examples

### EXAMPLE 8

### O/W Cream According to Table 6

TABLE 6
O/W cream according to Example 8

| | |
|---|---|
| Polyglyceryl-3 methylglucose distearate | 3.00% |
| Glyceryl stearate | 2.00% |
| Cetearyl alcohol | 1.00% |
| Ethylhexyl stearate | 10.00% |
| Decyl oleate | 9.00% |
| Silicone methacrylate particles from Ex. 1 | 1.50% |
| Glycerol | 3.00% |
| Water | ad 100% |
| Sodium benzoate, potassium sorbate, | q.s. |
| phenoxyethanol, perfume | |

### EXAMPLE 9

### Impregnation Solution for Wet Wipes According to Table 7

TABLE 7
Impregnation solution for wet wipes according to Example 9

| | |
|---|---|
| TEGO ® Wipe Flex | 5.70% |
| (Ethylhexyl stearate, phenoxyethanol, sorbitan | |
| laurate, polyglyceryl-4 laurate, dilauryl citrate) | |
| Cyclomethicone | 2.00% |
| Silicone methacrylate particles from Ex. 1 | 2.00% |
| Water | ad 100% |
| Glycerol | 3.00% |
| TEGO ® Carbomer 141 | 0.10% |
| Sodium hydroxide (10% in water) | q.s. |

TEGO ® Wipe Flex (Evonik Goldschmidt GmbH)
TEGO ® Carbomer 141 (Evonik Goldschmidt GmbH)

The impregnation solution can be used with the help of customary impregnation or spraying processes for producing cosmetic wet wipes (e.g. for baby care, make-up removers, cleansing wipes). For this, typically nonwovens are used, which generally have fibres of polyolefins, polyesters, celluloses, rayon, polyamides, polyesteramides or polyvinyl alcohols or consist of these or which are composed of mixed fibres of these components.

### EXAMPLE 10

### Make-Up Powder Foundation According to Table 8

TABLE 8
Make-up powder foundation according to Example 10:

| | |
|---|---|
| Zinc stearate | 3.00% |
| Mica | 40.00% |
| Talc | 24.00% |
| Iron oxide | 5.00% |
| Silicone methacrylate particles from Ex. 1 | 10.00% |
| Titanium dioxide | 8.00% |
| Cetylethyl hexanoate | 2.00% |
| Squalane | 3.00% |
| Cetearylethyl hexanoate | 2.00% |
| Mineral oil (30 mPas) | 2.00% |
| PEG/PPG-4/12 dimethicone | 1.00% |
| Aluminium starch octenylsuccinate | q.s. |
| Iron oxide | q.s. |
| Perfume | q.s. |

### EXAMPLE 11

### Make-Up Foundation According to Table 9

TABLE 9
Make-up foundation according to Example 11

| | |
|---|---|
| Phenyltrimethicone | 14.00% |
| Ethylhexyl palmitate | 14.60% |
| Cetylethyl hexanoate | 5.00% |
| Carnauba wax | 4.70% |
| Stearoxydimethicone | 4.00% |
| PVP/eicosene copolymer | 1.00% |
| Cetylstearyl heptanoate | 2.85% |
| Covabead LH 85, polymethyl methacrylate particles | 3.00% |
| Silicon dioxide | 0.25% |
| Zinc oxide | 7.00% |
| Nylon-12 | 2.00% |
| Talc Covasil 4.05 | 9.50% |
| Acrylate copolymer | 2.00% |
| Silicone methacrylate particles from Ex. 1 | 3.00% |
| Aluminium starch octenylsuccinate | 9.50% |
| Iron oxide | 3.10% |
| Titanium dioxide (and) dimethicone | 14.50% |

### EXAMPLE 12

### Eye-Shadow Formulation According to Example 10

TABLE 10
Eye-shadow formulation according to Example 12

| Cyclomethicone | ad 100% |
|---|---|
| PPG-3 myristyl ether | 7.00% |
| *Polyglyceryl-4 isostearate; cetyl PEG/PPG-10/1 dimethicone; | 1.00% |
| hexyl laurate | |
| Dimethicone (20 mPas) | 2.50% |
| Cera Alba | 4.50% |
| Carnauba wax | 2.00% |
| A-C copolymer 400 (ethylene/VA copolymer) | 2.50% |
| Ozokerite | 5.80% |
| C18-36-acid triglyceride | 2.00% |
| Liquapar oil (isobutylparaben (and) isopropylparaben (and) | 0.20% |
| butylparaben) | |
| Silicone methacrylate particles from Ex. 1 | 4.00% |
| Titanium dioxide | 5.00% |
| Chromium oxide green | 10.00% |
| CI 77491 (and) aluminium powder (and) silicon dioxide | 5.00% |
| CI 77891 (and) CI 77288 (and) mica | 10.00% |

*ABIL ® WE 09 (Evonik Goldschmidt GmbH)

### EXAMPLE 13

### O/W Sunscreen Lotion According to Table 11

TABLE 11
O/W sunscreen lotion according to Example 13

| | |
|---|---|
| Glyceryl stearate citrate | 3.00% |
| Cetearyl alcohol | 1.00% |
| Cetyl dimethicone | 0.20% |
| C₁₂-C₁₅ alkyl benzoate | 4.80% |
| Triisostearin | 1.00% |
| Diethylhexyl carbonate | 6.00% |
| *Titanium dioxide; trimethoxycaprylylsilane | 3.00% |
| Tocopheryl acetate | 0.50% |
| Ethylhexyl methoxycinnamate | 5.00% |
| Butyl methoxydibenzoylmethane | 2.50% |
| Carbomer | 0.20% |
| Xanthan | 0.40% |
| Sodium carboxymethyl betaglucan | 0.10% |
| Glycerol | 2.00% |
| Water | ad 100% |
| Silicone methacrylate particles from Ex. 1 | 1.50% |
| Sodium hydroxide (10% in water) | q.s. |
| Perfume | q.s. |

*Tego ® Sun T 805 (Evonik Goldschmidt GmbH)

### EXAMPLE 14

### Antiperspirant/Deodorant Roll-on According to Table 12

TABLE 12
Formulation according to Example 14

| | |
|---|---|
| Steareth-2 | 2.20% |
| Steareth-20 | 1.00% |
| Cetearyl ethylhexanoate | 2.00% |
| PPG-11 stearyl ether | 2.00% |
| Dimethicone | 0.50% |
| Polyglyceryl-3 caprylate | 0.50% |
| Aluminium chlorohydrate | 5.00% |
| Water | ad 100% |
| Glycerol | 3.00% |
| Silicone methacrylate particles from Ex. 1 | 0.30% |
| Perfume | q.s. |
| Citric acid (50% in water) | q.s. |
| Phenoxyethanol, ethylhexylglycerol | q.s. |

### EXAMPLE 15

### Lipstick According to Table 13

TABLE 13
Formulation according to Example 15

| | |
|---|---|
| Cyclopentasiloxane | 34.00% |
| Behenoxydimethicone | 3.00% |
| Stearyl dimethicone | 10.00% |
| Polyisobutene | 5.00% |
| Phenyl trimethicone | 8.00% |
| Isododecane | 4.00% |
| bis-Diglyceryl polyacyladipate-2 | 4.00% |
| Ceresin | 24.00% |
| Titanium dioxide | 1.00% |
| Carmine red | 1.00% |
| D&C Red No. 7 | 3.00% |
| Polyethylene | 1.00% |
| Silicone methacrylate particles from Ex. 1 | 1.00% |
| Aluminium starch octenylsuccinate & lauroyl lysine | 1.00% |

### EXAMPLE 16

### Mascara Formulation According to Table 14

TABLE 14
Formulation according to Example 16

| | |
|---|---|
| Sucrose stearate | 4.00% |
| Polyglyceryl-3 methylglucose distearate | 2.00% |
| Stearyl alcohol | 1.00% |
| Candelilla wax | 5.00% |
| Carnauba wax | 1.75% |
| Beeswax | 4.25% |
| Hydrogenated rice bran wax | 5.00% |
| Adipic acid/diethylene glycol/glycerol crosspolymer | 5.00% |
| Ceramide NP | 0.05% |
| Iron oxide | 10.00% |
| Silicone methacrylate particles from Ex. 1 | 0.50% |
| Water | 49.55% |
| 1,3-Butanediol | 3.00% |
| Triethanolamine | 1.80% |
| Acrylate/octylacrylamide copolymer | 5.00% |
| Phenoxyethanol; methylparaben; ethylparaben, butylparaben; | 0.60% |
| propylparaben, isobutylparaben | |
| Phenoxyethanol | 0.50% |

### EXAMPLE 17

### Make-Up Foundation According to Table 15

TABLE 15
Formulation according to Example 17

| | |
|---|---|
| *Bis(Glyceryl/lauryl) glyceryl/lauryl dimethicone | 4.00% |
| **Diethylhexyl carbonate | 3.10% |
| ***Phenoxyethyl caprylate | 3.10% |
| Mineral oil | 6.30% |
| Titanium dioxide | 4.00% |
| Iron oxide | 2.50% |
| Talc | 2.00% |
| Dicaprylyl carbonate (and) stearalkonium hectorite (and) | 2.00% |
| propylene carbonate | |
| Silicone methacrylate particles from Ex. 1 | 1.00% |
| ****Nylon-1010 | 1.00% |
| Dimethicone; dimethicone/vinyl dimethicone crosspolymer | 4.00% |
| Glycerol | 4.00% |
| Magnesium sulphate heptahydrate | 1.50% |
| Water | 60.80% |
| Methylparaben; ethylparaben; propylparaben; n-propylparaben; | 0.70% |
| Phenoxtol | |

*ABIL ® EM 120 (Evonik Industries AG)
**TEGOSOFT ® DEC (Evonik Industries AG)
***TEGOSOFT ® XC (Evonik Industries AG)
****TEGOLON ® ECO 10-10 (Evonik Industries AG)

### EXAMPLE 18

### Shower Gel Formulation According to Table 16

TABLE 16
Formulation according to Example 18

| | |
|---|---|
| *Acrylates/C10-30 alkyl acrylate crosspolymer | 1.60% |
| Water | 63.30% |
| Sodium lauryl sulphate | 21.40% |
| **Cocamidopropyl betaine | 5.30% |
| ***Polyglyceryl-3 caprate | 0.50% |
| Silicone methacrylate particles from Ex. 1 | 2.00% |
| Sodium hydroxide (10% in water) | q.s. |
| Phenoxyethanol; methylisothiazolinone | q.s. |
| Perfume | q.s. |

*TEGO ® Carbomer 341 ER (Evonik Industries AG)
**TEGO ® Betain F 50 (Evonik Industries AG)
***TEGOSOFT ® PC 31 (Evonik Industries AG)

### EXAMPLE 19

### Conditioner Formulation According to Table 17

TABLE 17
Formulation according to Example 19

| | |
|---|---|
| Water | 89.00% |
| *Cetrimonium chloride | 2.00% |
| **Behentrimonium chloride | 2.00% |
| ***Cyclopentasiloxane; dimethiconol | 1.00% |
| Silicone methacrylate particles from Ex. 1 | 0.50% |
| ****Cetearyl alcohol | 5.00% |
| Phenoxyethanol; methylisothiazolinone | q.s. |
| Perfume | q.s. |

*VARISOFT ® 300 (Evonik Industries AG)
**VARISOFT ® BT 85 (Evonik Industries AG)
***ABIL ® OSW 5 (Evonik Industries AG)
****TEGO ® Alkanol 1618 (Evonik Industries AG)

### EXAMPLE 20

### 2-In-1-Shampoo Formulation According to Table 18

TABLE 18
Formulation according to Example 20

| | |
|---|---|
| Sodium lauryl sulphate | 60.00% |
| Sodium cumenesulphonate | 3.00% |
| Water | 21.25% |
| *Cocamidopropyl betaine | 8.00% |
| **Cocamid MEA | 1.50% |
| ***Glycol distearate | 1.50% |
| ****Cetyl alcohol | 0.50% |
| Dimethicone (1000 mPa * s) | 1.50% |
| Silicone methacrylate particles from Ex. 1 | 1.00% |
| *****Nylon-1010 | 0.50% |
| Xanthan gum | 0.75% |
| Phenoxyethanol; methylisothiazolinone | q.s. |
| Perfume | q.s. |

*TEGO ® Betain F 50 (Evonik Industries AG)
**REWOMID ® C 212 (Evonik Industries AG)
***TEGIN ® G 100 (Evonik Industries AG)
****TEGO ® Alkanol 16 (Evonik Industries AG)
*****TEGOLON ® ECO 10-10 (Evonik Industries AG)

### EXAMPLE 21

### Styling Wax Formulation According to Table 19

TABLE 19
Formulation according to Example 21

| | |
|---|---|
| Water | 48.00% |
| Propylene glycol | 2.00% |
| Glycerol | 11.00% |
| *Methoxy PEG/PPG-7/3 aminopropyl dimethicone | 0.50% |
| **Isosteareth-20 | 14.50% |
| ***Laureth-4 | 10.00% |
| Paraffinum Perliquidum | 6.00% |
| ****C12-15 alkyl benzoate | 6.00% |
| Silicone methacrylate particles from Ex. 1 | 1.00% |
| Phenoxyethanol; methylisothiazolinone | q.s. |
| Perfume | q.s. |

*ABIL ® Soft AF 100 (Evonik Industries AG)
**REWODERM ® 66 E 20 (Evonik Industries AG)
***TEGO ® Alkanol L 4 (Evonik Industries AG)
****TEGOSOFT ® TN (Evonik Industries AG)

### EXAMPLE 22

### Leave-in Conditioner Formulation According to Table 20

TABLE 20
Formulation according to Example 22

| | |
|---|---|
| *Ceteareth-25 | 4.00% |
| **Cyclopentasiloxane; dimethiconol | 16.00% |
| ***Methoxy PEG/PPG-7/3 aminopropyl dimethicone | 1.00% |
| Silicone methacrylate particles from Ex. 1 | 3.00% |
| ****Laureth-4 | 0.50% |
| *****Carbomer | 0.50% |
| Water | 67.00% |
| Propylene glycol | 5.00% |
| Sodium hydroxide (10% in water) | ad pH |
| | 5-6 |
| Perfume | q.s. |
| Phenoxyethanol; methylisothiazolinone | q.s. |

*TEGINACID ® C (Evonik Industries AG)
**ABIL ® OSW 5 (Evonik Industries AG)
***ABIL ® Soft AF 100 (Evonik Industries AG)
****TEGO ® Alkanol L 4 (Evonik Industries AG)
*****TEGO ® Carbomer 140 (Evonik Industries AG)

1. A cosmetic composition comprising solid particles of silicone methacrylate which are obtained by
a) producing an emulsion of water and an organic phase, where the organic phase comprises organopolysiloxanes modified in a terminal and/or lateral position with methacrylate groups according to formula (I) or mixtures thereof 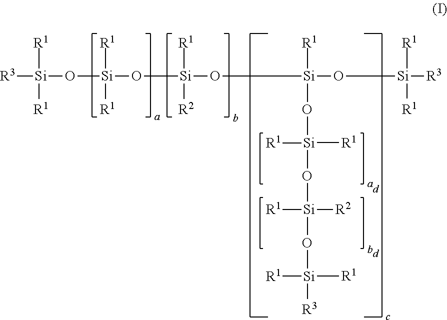
where
R¹=identical or different radicals, selected from linear or branched, saturated, mono- or polyunsaturated, linear, cyclic or branched alkyl, alkoxy, polyalkoxy, hydroxyalkyl, hydroxyalkoxy, alkenyl, aryl, aryloxy, hydroxyaryl, hydroxyaryloxy, alkaryl, alkaryloxy, hydroxyalkaryl, hydroxyalkaryloxy, aralkyl, aralkoxy, hydroxyaralkyl or hydroxyaralkoxy radicals having 1 to 20 carbon atoms,
R²=identical or different divalent hydrocarbon radicals having 1 to 20 carbon atoms bonded to the Si atom via an Si—C linkage and to which at least one methacrylic acid unit is attached via an ester bond,
R³=identical or different radicals R¹ or R²,
a=50 to 1000,
b=0 to 15,
c=0 to 5,
a_{d}=0 to 1000,
b_{d}=0 to 15,
where
the index d when c>0 is an integer>0,
with the proviso that when b and c=0, R³ is not selected from the same group as R¹, with the addition of at least one emulsifier, where the organic phase forms an internal phase of the emulsion, and
b) fully polymerizing the internal phase in the presence of a radical initiator, wherein said radical initiator is added to the external phase in a concentration of from 0.1 to 40% by weight based on the internal phase.

2. The composition according to claim 1, wherein said solid particles of silicone methacrylate have a maximum of particle size distribution in a range from 1 to 50 μm.

3. The composition according to claim 1, wherein the solid particles of silicone methacrylate are present in said composition in an amount from 0.1 to 10% by weight.

4. The composition according to claim 1, further comprising from 0.1 to 20% by weight based on the total composition, of components which absorb or filter out UV radiation.

5. The composition according to claim 1, further comprising from 0.1 to 15% by weight of cosmetic particles which are different from said solid particles of silicone methacrylate.

6. The composition according to claim 1, further comprising from 0.1 to 20% by weight of organosilicon compounds.

7. The composition according to claim 1, wherein said emulsifiers are particulate emulsifiers selected from the group of metal oxides, mixed oxides, nitrides, hydroxides, carbonates, silicates, silicone resins, silicones and/or organic polymers which are at least partially hydrophobicized and with at least one compound from the group of silanes, siloxanes, quaternary ammonium compounds, cationic polymers and fatty acids or anions thereof.

8. The composition according to claim 1, wherein more than 95 mol % of the radicals R¹ in formula (I) are methyl groups.

9. The composition according to claim 1, wherein R² in formula (I) is selected from the group of radicals 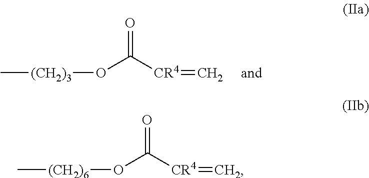
where R⁴ is a methyl group.

10. The composition according to claim 1, wherein a of formulation (I) is from 150 to 210, b of formula (I) is from 3 to 9, and c of formula (I) is equal to 0.

11. A method of forming a cosmetic composition, said method comprising providing solid particles of silicone methacrylate by
a) producing an emulsion of water and an organic phase, where the organic phase comprises organopolysiloxanes modified in a terminal and/or lateral position with methacrylate groups according to formula (I) or mixtures thereof, 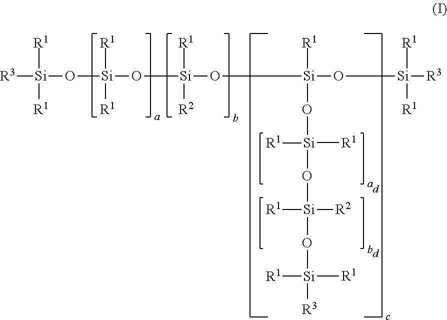
where
R¹=identical or different radicals, selected from linear or branched, saturated, mono- or polyunsaturated, linear, cyclic or branched alkyl, alkoxy, polyalkoxy, hydroxyalkyl, hydroxyalkoxy, alkenyl, aryl, aryloxy, hydroxyaryl, hydroxyaryloxy, alkaryl, alkaryloxy, hydroxyalkaryl, hydroxyalkaryloxy, aralkyl, aralkoxy, hydroxyaralkyl or hydroxyaralkoxy radicals having 1 to 20 carbon atoms,
R²=identical or different divalent hydrocarbon radicals having 1 to 20 carbon atoms bonded to the Si atom via an Si—C linkage and to which at least one methacrylic acid unit is attached via an ester bond,
R³=identical or different radicals R¹ or R²,
a=50 to 1000,
b=0 to 15,
c=0 to 5,
a_{d}=0 to 1000,
b_{d}=0 to 15,
where
the index d when c>0 is an integer>0,
with the proviso that when b and c=0, R³ is not selected from the same group as R¹, with the addition of at least one emulsifier, where the organic phase forms an internal phase of the emulsion, and
fully polymerizing the internal phase in the presence of a radical initiator, wherein said radical initiator is added to the external phase in a concentration of from 0.1 to 40% by weight based on the internal phase.

12. The method according to claim 11, wherein said solid particles of silicone methacrylate have a maximum of particle size distribution in a range from 1 to 50 μm.

13. The method according to claim 11, wherein the emulsifiers are particulate emulsifiers selected from the group of metal oxides, mixed oxides, nitrides, hydroxides, carbonates, silicates, silicone resins, silicones and/or organic polymers, which are at least hydrophobicized with at least one compound from the group of silanes, siloxanes, quaternary ammonium compounds, cationic polymers and fatty acids or anions thereof.

14. The method according to claim 11, wherein more than 95 mol % of the radicals R¹ in formula (I) are methyl groups.

15. The method according to claim 11, wherein R² in formula (I) is selected from the group of radicals 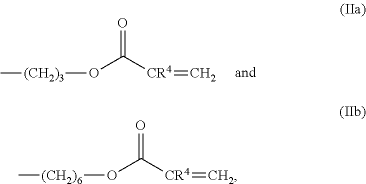
where R⁴ is a methyl group.

16. The method according to claim 11, wherein a of formula (I) is from 150 to 210, b of formula (I) is from 3 to 9, and c of formula (I) is equal to 0.
